(19) United States
(12) Patent Application Publication
(10) Pub. No.: US 2012/0006329 A1
(43) Pub. Date: Jan. 12, 2012

(54) METHOD OF PARTIALLY ADHERING CPAP MASK TO FACE

(76) Inventors: Katherine K. Beevers, McMinnville, OR (US); Timothy R. Beevers, McMinnville, OR (US)

(21) Appl. No.: 12/831,041

(22) Filed: Jul. 6, 2010

(51) Int. Cl. A61M 16/06

(52) U.S. Cl. 128/206.25

(57) ABSTRACT

A method of providing a good seal between a user's nose and a CPAP mask having a portion designed to contact the upper part of a user's nose, that makes use of a piece of double-sided lightly adhesive tape. The tape is placed about the upper part the user's nose and the CPAP mask is placed on the user's face, so that the tape contacts the portion designed to contact the upper part of user's nose.

### BACKGROUND

Those who suffer from sleep apnea, a significant portion of the American population, are frequently treated with continuous positive airway pressure (CPAP). This involves air being gently blown into the nose, by a machine that is engaged with the nose by way of an air tube attached to a mask that is fitted over the nose and held tightly in place with a system of straps that go about the head. Unfortunately, the air that is in the mask, which is at a higher pressure than the air about it, has a tendency to blow out of the mask at any place where the seal between the mask and the skin about the nose is not air-tight. This is particularly bothersome when the air blows out toward the eyes of the patient. As a result, those being treated with CPAP sometimes have a difficult time adjusting the straps so that they hold the mask to the face tightly enough to avoid bothersome leaks, but not so tight as to be so uncomfortable that the patient cannot sleep.

### SUMMARY

The following embodiments and aspects thereof are described and illustrated in conjunction with systems, tools and methods which are meant to be exemplary and illustrative, not limiting in scope. In various embodiments, one or more of the above-described problems have been reduced or eliminated, while other embodiments are directed to other improvements.

In a first separate aspect, the present invention may take the form of a method of providing a good seal between a user's nose and a CPAP mask having a portion designed to contact the upper part of a user's nose, that makes use of a piece of double-sided lightly adhesive tape. The tape is placed about the upper part the user's nose and the CPAP mask is placed on the user's face, so that the tape contacts the portion designed to contact the upper part of user's nose.

In a second separate aspect, the present invention may take the form of a CPAP mask having skin contacting portions that are lightly adherent, so that they gently adhere to a patient's skin when the mask is in use.

In a third separate aspect, the present invention may take the form of a CPAP mask wearing facilitating assembly, comprising a U-shaped piece of tape having a first adherent side and a second adherent side, packaged between a first and second sheet of backing material. To use the assembly, the first piece of backing material may be removed, exposing the first adherent side, and the first adherent side may be adhered to a first surface, and then the second piece of backing material may be removed and the second adherent side may be adhered to a second surface.

In addition to the exemplary aspects and embodiments described above, further aspects and embodiments will become apparent by reference to the drawings and by study of the following detailed descriptions.

### BRIEF DESCRIPTION OF THE DRAWINGS

FIG. 1 is a perspective view of a patient wearing a CPAP according to the method of the present invention. The straps retaining the CPAP mask to the patients face have been omitted for ease of presentation.

FIG. 2 is a sectional view of a patients nose, adherent strip and CPAP mask.

FIG. 3 is an exploded view of nose tissue, an adherent strip and CPAP mask material.

FIG. 4 is a plan view of adherent strip used in the method shown in FIG. 1.

Exemplary embodiments are illustrated in referenced drawings. It is intended that the embodiments and figures disclosed herein are to be considered illustrative rather than restrictive.

### DETAILED DESCRIPTION OF THE PREFERRED EMBODIMENTS

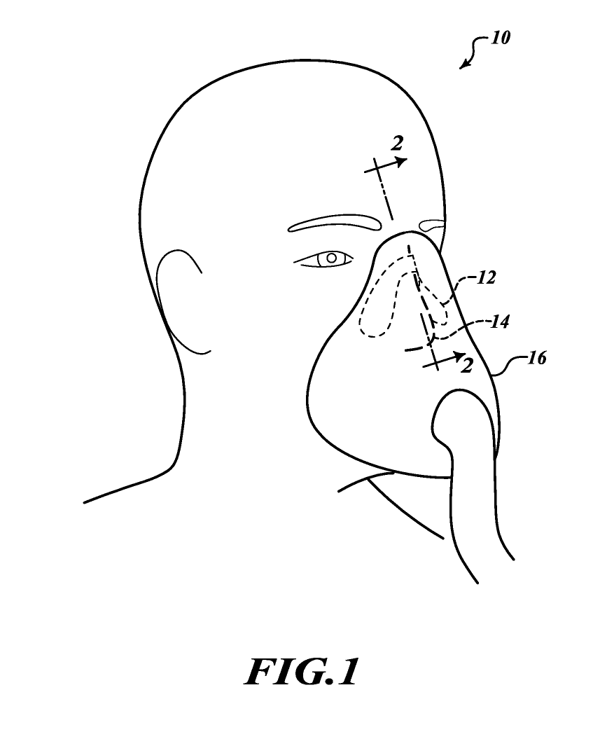

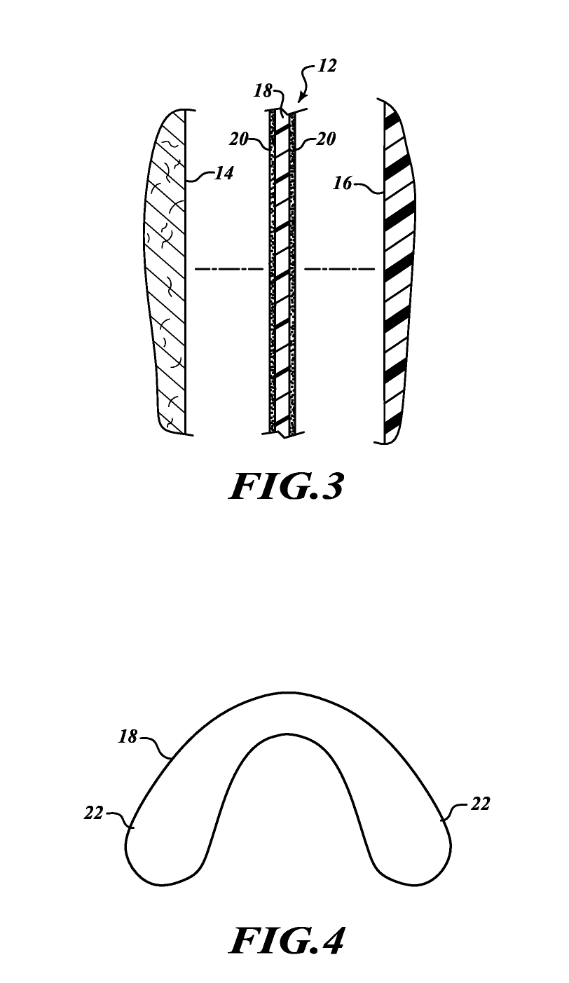

Referring to FIGS. 1, 2 and 3, one preferred embodiment of the present invention is a method in which a CPAP mask user 10, places a U-shaped piece of double sided, lightly adhesive tape 12 between his nose 14 and a CPAP mask 16. Tape piece 12, is composed of a central membrane 18, covered on either side by a layer of lightly adhesive material 20. A central bridge portion 22 is designed to adhere the CPAP mask to the bridge of the nose 14, whereas wings 24 act to adhere the mask to the upper sides of the nose 14. Altogether, tape piece 12 prevents air from blowing out of the volume contained between mask 16 and the face. Air escaping about the upper portion of the nose 14 is particularly troublesome, because it tends to blow toward the eyes.

In a preferred embodiment, tape piece 12 is disposable, with a fresh piece being used every night. In this embodiment, boxes containing between 10 and 100 or units of tape piece 12 would be sold. Each piece 12 is provided between two backing sheets, permitting a user to remove a first backing sheet and adhere tape piece 12 to the nose, then remove the other backing sheet and put on the mask 16, taking care to adhere the portion that covers the upper part of the nose 14, to tape piece 12. Alternatively, the patient adheres tape piece 12 first to the mask 16 and then places the mask over his face, with the tape piece 12 adhering to the top part of the nose 14.

In a preferred embodiment, central membrane 18 is a thin (0.2 mm, for example) sheet of clear polymeric material such as cellophane or polyimide. In an alternative preferred embodiment membrane 18 is thicker (1 mm, for example) and made of material that is resiliently compressible to give the user some cushioning effect. For example the material used for “Flent's Nose Pads for Eyeglass Wearers,” may be used for membrane 18. The membrane material may also be absorbent, to absorb the user's skin oil while he sleeps.

The material of layers 20 may be selected from a wide range of materials such as a gently adhesive hydrocolloid, a silicone or an acrylic adhesive. Any material chosen must, of course be biocompatible and non-irritating to human skin. For example, material no. PS-1243 available from Bluestar Silicones, which maintains a website having address http://www.bluestarsilicones.com, may be used. Although both layers 20 are designated with the same reference number, they may be composed of mutually distinct adhesive material.

In a preferred embodiment tape piece 12 is used in conjunction with a mask 16 designed to interact with tape piece 12. Currently, masks such as mask 16 are designed to create a gentle pressure seal all about the nose. If an adhesive tape piece 12 is used, however, the portion contacting the top of the nose need not exert pressure. This opens up the design of masks 16 to a design that creates a pressure seal elsewhere, but in the upper nose region is designed to engage with tape piece 12. In this region, a material adapted to adhere readily is used in the mask area designed to engage with the upper nose.

In an alternative preferred embodiment, only a first side of tape piece 12 is adherent, while the other side is designed to mechanically engage with a mating mask area. In one embodiment a plastic zipper, similar to those used in food storage bags is used to engage the mask to tape piece 12. In another embodiment hooks are used to mate the two, and a plastic flap makes the seal air tight.

In an additional preferred embodiment, tape pieces are used for other parts of the face where air is escaping, either in addition to or instead of tape piece 12, positioned about the upper nose.

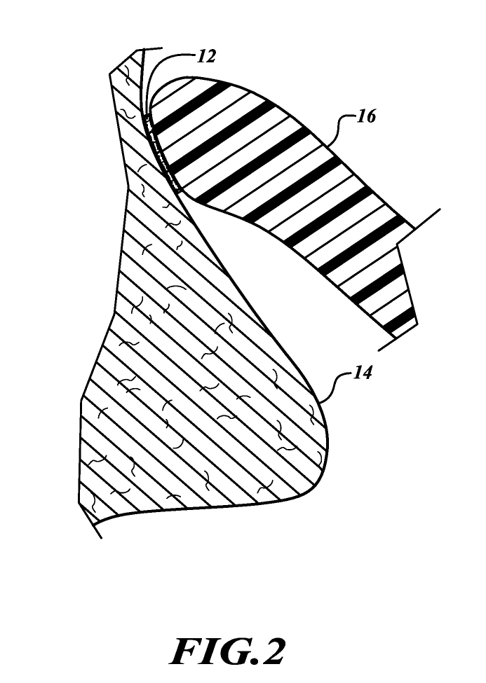

In a preferred embodiment the region marked with the reference number 12 in FIG. 2 is not an adherent tape piece, but rather an adherent region of mask 16, itself In another embodiment adherent region 12 is formed of a material that is adherent, yet washable. For example the Blue Star silicone material noted above as a possible adhesive for use in layer 20, could be used in this embodiment, also. In this embodiment, skin oil and detritus can be washed off of region 12 between uses.

While a number of exemplary aspects and embodiments have been discussed above, those possessed of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope.

1. A method of providing a good seal between a user's nose and a CPAP mask having a portion designed to contact the upper part of a user's nose, comprising:
(a) providing a piece of double-sided lightly adhesive tape;
(b) placing said piece of tape about the upper part said user's nose;
(c) placing said CPAP mask on said user's face, so that said tape contacts said portion designed to contact the upper part of user's nose.

2. The method of claim 1, wherein said tape is disposed of after one use.

3. The method of claim 1, wherein said tape is U-shaped, with the center of the U being fit over the bridge of the nose and each arm of the U descending about a side of the nose.

4. The method of claim 1, wherein said portion of said mask designed to contact the upper part of a user's nose is adapted to adhere to said tape.

5. The method of claim 1, wherein said adhesive tape includes a central membrane and an adhesive layer on either side.

6. The method of claim 5, wherein said central membrane is made of resiliently compressible material.

7. The method of claim 5, wherein said central membrane is made of absorbent material and absorbs some of the body oil secreted during mask use.

8. The method of claim 5, wherein said central membrane is a thin sheet of clear polymeric material.

9. The method of claim 1, wherein said double sided lightly adhesive tape includes an adhesive layer made of hydrocolloid.

10. The method of claim 1, wherein said double sided lightly adhesive tape includes an adhesive layer made of silicone.

11. The method of claim 1, wherein said double sided lightly adhesive tape includes an adhesive layer made of an acrylic adhesive.

12. A CPAP mask having skin contacting portions that are lightly adherent, so that they gently adhere to a patient's skin when said mask is in use.

13. The CPAP mask of claim 12, wherein said portions that are lightly adherent are made of a material that may be easily washed of oils and particles.

14. The CPAP mask of claim 12, wherein said portions that are lightly adherent are made of a biocompatible silicone adhesive.

15. A CPAP mask wearing facilitating assembly, comprising a U-shaped piece of tape having a first adherent side and a second adherent side, packaged between a first and second sheet of backing material, so that a first piece of backing material may be removed, exposing said first adherent side, and said first adherent side may be adhered to a first surface, and then said second piece of backing material may be removed and said second adherent side may be adhered to a second surface.

16. The assembly of claim 15, further comprising additional, identical U-shaped pieces of tape, packaged in a sealed cardboard box.

17. The assembly of claim 16, wherein said sealed cardboard box contains between 5 and 15 identical U-shaped pieces of tape.

18. The assembly of claim 16, wherein said sealed cardboard box contains between 15 and 150 identical U-shaped pieces of tape.

19. The assembly of claim 15, wherein one of said adherent sides is made of a hydrocolloid.

20. The assembly of claim 15, wherein one of said adherent sides is made of a biocompatible silicone adhesive.
